(19) United States
(12) Plant Patent Application Publication
(10) Pub. No.: US 2001/0047529 P1
(43) Pub. Date: Nov. 29, 2001

(54) Anthirrhinum plant named Balumpink

(76) Inventor: Scott Trees, Shell Beach, CA (US)

Correspondence Address: ROCKEY, MILNAMOW & KATZ, LTD., Two Prudential Plaza, 180 North Stetson Avenue, Suite 4700, Chicago, IL, 60601, US

(21) Appl. No.: 09/808,532

(22) Filed: Mar. 14, 2001

(30) Foreign Application Priority Data
Mar. 23, 2000 (CA) 00-2182

(51) Int. Cl. A01H 5/00

(52) U.S. Cl. PLT/322

(57) ABSTRACT

A new and distinct Antirrhinum plant named Balumpink characterized by its trailing habit, dark green glabrous foliage and pink flowers.

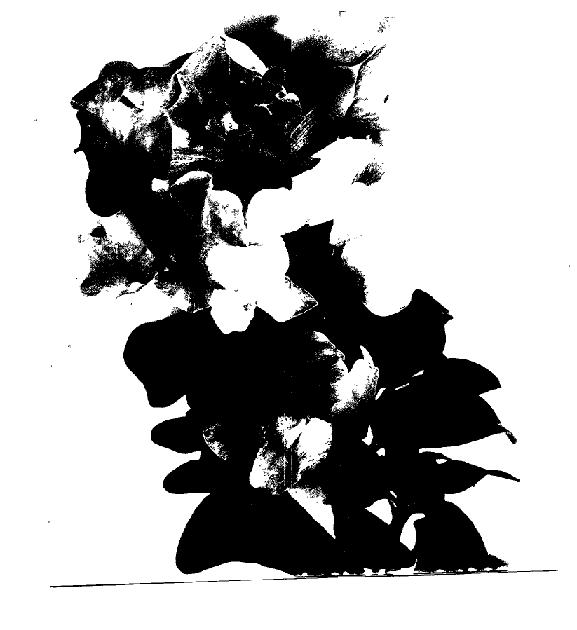

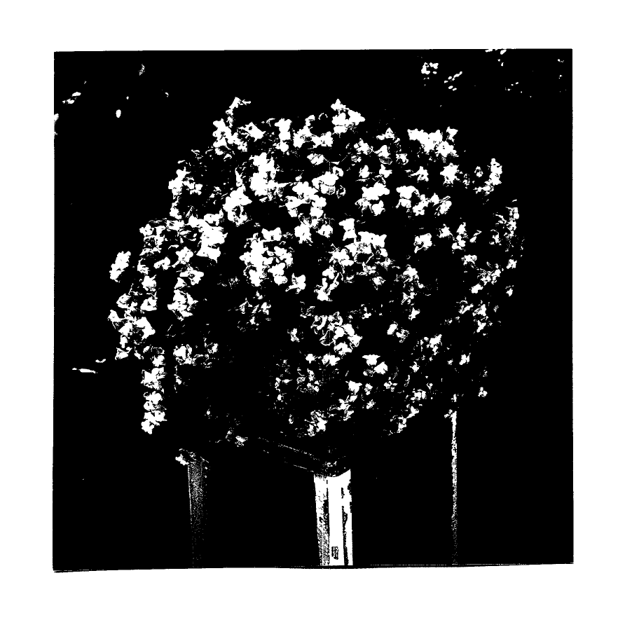

### BACKGROUND OF THE INVENTION

The present invention comprises a new and distinct Antirrhinum plant, hereinafter referred to by the cultivar name Balumpink. This new cultivar was developed by the inventor through a controlled breeding program during August 1997 at Arroyo Grande, Calif. The objective of the breeding program was the development of Antirrhinum cultivars with trailing mounded habit, continuous flowering, excellent basal branching and small, dark green foliage.

Balumpink resulted from the self fertilization of the proprietary antirrhinum plant designated 90-2 which is characterised by its compact habit, purple flowers and medium green glabrous foliage. The resulting seed was collected and germinated. From the flowering progeny, a plant was selected in April 1998 and initially designated 229-2.

Asexual reproduction of the new cultivar by terminal or stem cuttings taken during 1998 and 1999 at Arroyo Grande, Calif., U.S.A. has demonstrated that the characteristics of the new cultivar as herein described are firmly fixed and retained through successive generations of such asexual propagation.

### SUMMARY OF THE INVENTION

It was found that the cultivar of the present invention:

(a) exhibits a trailing habit,

(b) forms abundant bright pink flowers and

(c) exhibits dark green foliage.

When the new cultivar of the present invention is compared to Candelabra Rose Pink (not patented in the United States) it is found that the new cultivar exhibits a somewhat less trailing habit, larger foliage with a different texture and flowers that differ in color and fragrance as detailed in TABLE A.

### BRIEF DESCRIPTION OF PHOTOGRAPHS

The accompanying photographs show as nearly true as it is reasonably possible to make the same in color illustrations of this type, typical flower and foliage characteristics of the new cultivar.

The first photograph comprises a view of the growth habit of Balumpink in a pot containing four plants.

The second photo comprises a close-up view of typical flowers and foliage of Balumpink. Colors are darker than the actual flower and foliage colors.

The plants were grown in a greenhouse at West Chicago, Ill., USA.

### DETAILED BOTANICAL DESCRIPTION

The Balumpink cultivar has not been observed under all possible environmental conditions to date. Accordingly, it is possible that the phenotype may vary somewhat with variations in the environment, such as temperature, light intensity and day length.

The chart used in the identification of colors described herein is the R.H.S Colour Chart of The Royal Horticultural Society, London, England. The color values were determined on Sep. 26, 2000 in West Chicago, Ill. The readings were taken between 11:00 and 11:45 p.m. under natural light conditions. The plants were produced from cuttings taken from stock plants and grown for ten (10) weeks in a double poly carbonate covered greenhouse under conditions comparable to those used in commercial practice while utilizing a soilless growth medium and maintaining temperatures of approximately 72° F. during the day and approximately 65° F. during the night.

Classification:

Botanical—Antirrhinum majus cultivar Balumpink

Commercial—Trailing Snapdragon

Propagation:

Type cutting—Terminal tip

Time to Initiate Roots—Approximately 10-14 days with the shorter times generally being experienced in the summer and the longer times in the winter

Root Description—Fibrous, branching

Plant Description:

General Appearance and Form—Mounded and trailing

Plant length (from top of plant plane to lower flowers)—Approximately 16 cm p1 Foliage width—Approximately 28 cm

Branching habit—Freely branching. Pinching encourages branching.

Stem—Diameter at lowest node approximately 5 mm, closest to 146B in color, smooth in texture

Internode length—Approximately 2.2 cm

Foliage description:

Shape—Ovate, acute apex, attenuate base, entire margin

Arrangement—Opposite

Venation pattern—Arcuate

Texture—Upper and lower surface: smooth

Color of mature foliage—Upper surface closest to 137A with venation of 146A, lower surface closest to 146A with venation of 146A

Size of mature leaves—Approximately 3.3 cm in length, approximately 1.8 cm in width

Petiole—Length is approximately 3 mm, diameter is approximatelyl mm, color is 146A, texture is smooth

Flower Description:

Flowering Habit—Balumpink is freely flowering under outdoor growing conditions with substantially continuous blooming from spring until fall.

Flower arrangement—Terminal racemes

Peduncle—Length is approximately 9 mm, diameter is approximately 1 mm, color is closest to 146D

Flower—Approximately 3.2 cm in length, bilabiate. Lip texture is smooth, margin entire and somewhat undulating. Upper lip is 2 parted, approximately 1.8 cm long, approximately 2.3 cm wide, 62A at margins fading to 62D where lip meets palate. Lower lip is 3 parted, approximately 1.3 cm in length, approximately 2 cm in width, 62A at edges fading to 62D with two areas of 6A where lip meets palate. The palate is 155D and covered with hairs of 155D. Inside throat is 155D with hairs of 6A and venation of 63C, outside throat is between 62C and 62D.

Flower bud—Length is approximately 1.9 cm, width is approximately 9mm, texture is pilose

Calyx—5 sepals, approximately 8 mm in length, approximately 4 mm in width, ovate with acute apex, surface is pilose, upper and lower surface color is closest to 137C

Reproductive Organs—Androecium: 4 stamens, 2 are 1.8 cm in length, 155D; 2 are 1.4 cm in length 155D with slight anthocyanin coloration. Anthers approximately 2 mm in length, color is 12D. Pollen color is closest to 12A. Gynoecium: one pistil. Stigma length is approximately 2 mm, color is 144B. Style is approximately 1.4 cm in length, color is 144D with slight anthocyanin coloration. Ovary is 2 mm in diameter and hoary.

Seed production: Seed production has not been observed.

Disease resistance: Resistance to pathogens is not claimed.

TABLE A

| | | |
|---|---|---|
| CHARACTERISTIC | BALUMPINK | CANDELABRA |
| | | ROSE PINK |
| PLANT WIDTH-CM | 28 CM | 31.8 CM |
| MATURE LEAF LENGTH × | 3.3 × 1.8 | 1.2 × 1 |
| WIDTH-CM | | |
| LEAF SHAPE | OVATE | OVAL |
| LEAF TEXTURE | GLABROUS | PUBESCENT |
| FLOWER COLOR | 62A | 72B |
| FLOWER FRAGRANCE | NONE | SWEET |
| FLOWER LENGTH × | 3.4 × 1.7 | 3.3 × 1.6 |
| WIDTH OF | | |
| LOWER LIP-CM | | |

I claim:

1. A new and distinct cultivar of Antirrhinum named ‘Balumpink’ substantially as herein shown and described, which:
(a) exhibits a trailing growth habit,
(b) forms abundant bright pink flowers and
(c) exhibits dark green foliage.
